(19) United States
(12) Patent Application Publication
(10) Pub. No.: US 2004/0058374 A1
(43) Pub. Date: Mar. 25, 2004

(54) Method for mixing nucleic acid with a water insoluble medium and appalication thereof

(76) Inventors: Jun-Jei Sheu, Chungho City (TW); Ming-Hwa Liang, Chungho City (TW); Chung-Shung Chen, Chungho City (TW)

Correspondence Address: BIRCH STEWART KOLASCH & BIRCH, PO BOX 747, FALLS CHURCH, VA, 22040-0747, US

(21) Appl. No.: 10/645,602

(22) Filed: Aug. 22, 2003

(30) Foreign Application Priority Data
Aug. 30, 2002 (JP) 2002-294229

(51) Int. Cl. C12Q 1/68; B05D 3/00

(52) U.S. Cl. 435/6; 427/2.11

(57) ABSTRACT

The present invention relates to a method for mixing nucleic acid with a water insoluble medium and application thereof, specifically to a method for mixing nucleic acid solution in a water insoluble medium through the addition of an intermediate solution. The medium is an inert medium and is not deteriorative to nucleic acid, and the intermediate solution increases solubility between the nucleic acid solution and the water insoluble medium. The resulted water-insoluble medium containing nucleic acid is used to label solid or liquid substances or articles.

### BACKGROUND OF THE INVENTION

1. Field of the Invention

The present invention relates to a method for mixing nucleic acid with a water insoluble medium and application thereof, specifically to a method for mixing nucleic acid solution in a water insoluble medium through the addition of an intermediate solution.

### The Prior Arts

With the development of biotechnology, the application of biotechnology is not limited to the research work in laboratory anymore. In medical field, the process of prevention, identification, and even the treatment of diseases need to combine with molecular biology skills to acquire the best results. Utilization of biological methods to improve the strains of the crops and the livestock are also conspicuous. Furthermore, through the combination with digital system, people now are able to transfer the individual unique features into digital signals. For example, switching on household appliances by the owner's voice and utilizing individual fingerprints or irises for identification. The application of biotechnology to daily life matters is an inevitable trend in the future.

Nucleic acids, Ribonucleic acid (RNA) and Deoxyribonucleic acid (DNA), serve as storage units for our hereditary information. RNA and DNA are long polymers consisted of only 4 nucleotides, adenine (A), guanine (G), cytosine (C) and thymine (T) for DNA (or uracil (U) for RNA). The nucleotide structure can be broken down into 2 parts, the sugar-phosphate backbone and the base. All nucleotides share the sugar-phosphate backbone. The 3′-hydroxyl group on the ribose unit, reacts with the 5′-phosphate group on it's neighbor to form a chain.

The base on each nucleotide is different, but they still show similarities. adenine (A) and guanine (G) are purines, consisted of two ring structure, with the differences in the molecules coming in the groups attached to the ring. Likewise, cytosine (C) and thymine (T) and uracil (U) are pyrimidines and share a similar structure, but differ in their side groups. A, T, G and C are capable of pairing together to form a double strand. Adenine forms two hydrogen bonds with thymine in DNA (uracil in RNA) and cytosine forms 3 with guanine. That is, T will bond to A only and G to C only.

Among nucleic acid, DNA is very long lasting and the modifiers and degraders are well known and uncommon in normal circumstances. Fossil evidence shows that DNA is resistant to degradation over millions of years and is being used to learn more about ancient people and animals. DNA is an extremely stable molecule and is thus ideal for use as an identification marker. In addition, the ability to perform downstream reactions on nucleic acid molecules, such as PCR, is not affected by subjecting nucleic acid to extreme conditions of heat, which is the great advantage of nucleic acid for labeling.

There are two major identification methods used nowadays. Except the unique features of the merchandise, another way is to label or mark the objects with specific labels. Traditional labels take advantage of physical or chemical properties of materials. For example, magnetic strips on checkbooks, laser holographs on credit cards, fluorescent ink on stocks, and heat-sensitive inks. However, those labels are easily to be mimicked, destroyed by illicit persons.

It is well-known to persons skilled in the related art that nucleic acid, a highly water-soluble molecule, easily dissolves in water-soluble solution, such as TE buffer. However, dissolving nucleic acid with water-insoluble solvents or medium seems not feasible. The labeling method in the related art is to dissolve DNA in water-soluble solution and spread on the target. However, DNA taggants are easily removed after drying in the related art. That is DNA taggants cannot adhere on the objects for a long period of time and lose its anti-counterfeiting function.

### SUMMARY OF THE INVENTION

Accordingly, the present invention is directed to a method for mixing nucleic acid with a water-insoluble medium and application thereof that substantially obviates one or more of the problems due to limitations and disadvantages of the related art.

A primary object of the present invention is to provide a method for mixing nucleic acid with a water-insoluble medium and application thereof, in which a nucleic acid solution is well mixed with a water-insoluble medium through the addition of an intermediate solution.

Another object of the present invention is to provide a method for labeling solid substances or articles, in which the target is spread with water-insoluble media containing specific nucleic acid.

Still object of the present invention is to provide a method for labeling liquid substances or articles, in which the target is mixed with water-insoluble media containing specific nucleic acid.

A further object of the present invention is to provide a water-insoluble medium containing nucleic acid, which is water insoluble and is capable of adhering to the objects.

In order to achieve the foregoing objects, a method for mixing nucleic acid with a water insoluble medium is provided. In the process, prepared nucleic acid is dissolved in a first solvent to form a first mixture. Water-insoluble medium is dissolved in a second solvent to form a second mixture. Then intermediate solution is added to the first mixture. The first mixture having intermediate solution is mixed with the second mixture to form a third mixture. The medium is an inert medium and is not deteriorative to nucleic acid. The intermediate solution increases solubility between the first mixture and the second mixture.

For more detailed information regarding advantages and features of the present invention, examples of preferred embodiments will be described below with reference to the annexed drawings.

### BRIEF DESCRIPTION OF THE DRAWINGS

The related drawing in connection with the detailed description of the present invention to be made later is described briefly as follows, in which:

FIG. 1 shows a flowchart of the process of mixing nucleic acid with a water insoluble medium of the invention.

### DETAILED DESCRIPTION OF THE PREFERRED EMBODIMENT

Preferred embodiments of the present invention will now be described in further detail. It should be understood that these examples are intended to be illustrative only and that the present invention is not limited to the conditions or materials recited therein.

The term “nucleic acid” used in the invention presents both deoxyribonucleic acid (DNA) and ribonucleic acid (RNA).

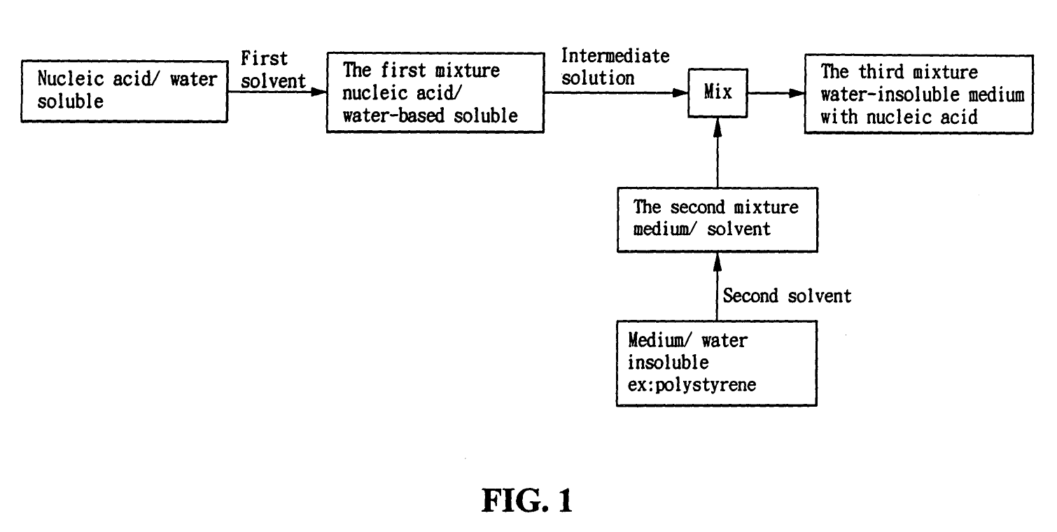

As shown in FIG. 1, the process of mixing nucleic acid with a water insoluble medium of the invention comprises at least the following steps. First, prepared nucleic acid is dissolved in a first solvent to form the first mixture, the nucleic acid/water-based solution. A water-insoluble medium is dissolved in a second solvent to form the second mixture, the medium/solvent mixture. Then intermediate solution is added to the first mixture. The first mixture having intermediate solution is mixed with the second mixture to form the third mixture, the water-insoluble medium containing nucleic acid. The intermediate solution increases solubility between the nucleic acid/water-based solution and the water-insoluble medium/solvent solution.

The medium is an inert medium and is not deteriorative to nucleic acid. The water-insoluble medium comprises a polymeric substance. As used herein, the polymeric substance is selected from a group consisting of polypropylene (PP), polycarbonate (PC) and polystyrene (PS). In one preferred embodiment, the water-insoluble medium is polystyrene (PS).

The second solvent used to dissolve the water-insoluble medium comprises an organic solvent. As used herein, the second solvent is selected from a group consisting of chloroform, dichloromethane and benzole solvent, such as xylene or toluene. However, other organic solvent known in the related art may also be used.

The nucleic acid used herein is selected from a group consisting of natural and synthetic nucleic acid. The term “natural nucleic acid” as used herein means nucleic acid prepared from all prokaryotes, eukaryotes, such as animals, plants, viruses, fungi and others. The term “synthetic nucleic acid” includes synthetic vectors and synthetic nucleic acid fragments.

The first solvent comprises a water-soluble solution. The water-soluble solution comprises water, TE buffer or PBS buffer.

The intermediate solution increases solubility between the nucleic acid/water-based solution and the water-insoluble medium/solvent solution. The polarity of the intermediate solution is between that of the first and second mixture. The intermediate solution comprises an organic solvent. The organic solvent is selected from a group consisting of ethanol, acetone and their mixture. The intermediate solution is added to a final concentration of between 5 and 50% of the water-insoluble medium.

For labeling solid substances or articles, the water-insoluble medium containing known nucleic acid is spread on the target solid substances or articles. After the medium containing known nucleic acid is dried, nucleic acid protected by the water-insoluble medium adheres on surface of the object.

For labeling liquid substances or articles, the target liquid is mixed with the water-insoluble media containing known nucleic acid. As a result, the target liquid substance or article is labeled with nucleic acid.

The above-mentioned solid substances or articles include antiques, paintings, jewelry, identification cards, credit cards, magnetic strip cards, sports collectibles, souvenirs and other solid collectibles. The foregoing liquid substances or articles include inks, paints, dyes, dyestuffs, color wash, pigments, seals, glues, cosmetics and others. After labeling with nucleic acid, the objects have anti-counterfeiting function.

Also, the water-insoluble medium containing nucleic acid could be used as materials to manufacture products with nucleic acid labeled.

### EXAMPLE

### Mixing DNA with Polystyrene (PS)

MATERIALS: DNA, water, polystyrene (PS), chloroform, 95% ethanol and acetone as the intermediate solution

5 μg of prepared DNA is dissolved in 100 μl of distilled water to form a DNA solution. 5 g of PS is dissolved in 50 ml chloroform to a concentration of 10% (wv). 10 μl of 95% ethanol and acetone, as intermediate solution, are added respectively to the DNA solution. Then, DNA solution containing the intermediate solution is mixed homogeneously with the PS solution through vigorous vortex. Through such intermediate process, water-soluble DNA solution and water-insoluble medium of PS solution are mixed completely to form the medium of PS solution containing the desired DNA.

While the invention has been described in its preferred embodiments, this should not be construed as limitation on the scope of the present invention. Accordingly, the scope of the present invention should be determined not by the embodiment illustrated, but by the appended claims and their legal equivalents.

What is claimed is:

1. A method for mixing nucleic acid with a water insoluble medium and application thereof, comprising the following steps:
dissolving nucleic acid in a first solvent to form a first mixture;
dissolving a water-insoluble medium in a second solvent to form a second mixture;
adding intermediate solution to the first mixture; and
mixing the first mixture having intermediate solution with the second mixture to form a third mixture;
wherein the medium is an inert medium and is not deteriorative to nucleic acid, and
wherein the intermediate solution increases solubility between the first mixture and the second mixture.

2. The method as claimed in claim 1, wherein the water-insoluble medium comprises a polymeric substance.

3. The method as claimed in claim 2, wherein the polymeric substance is selected from a group consisting of polypropylene (PP), polycarbonate (PC) and polystyrene (PS).

4. The method as claimed in claim 1, wherein the second solvent comprises an organic solvent.

5. The method as claimed in claim 4, wherein the organic solvent is selected from a group consisting of chloroform, dichloromethane or benzole solvent.

6. The method as claimed in claim 5, wherein the benzole solvent comprises xylene or toluene.

7. The method as claimed in claim 1, wherein the intermediate solution comprises an organic solvent.

8. The method as claimed in claim 7, wherein the organic solvent is selected from a group consisting of ethanol, acetone and their mixture.

9. The method as claimed in claim 1, wherein the intermediate solution is added to a final concentration of between 5 and 50% of the water-insoluble medium.

10. The method as claimed in claim 1, wherein the first solvent comprises a water-soluble solution.

11. The method as claimed in claim 10, wherein the water-soluble solution is selected from a group consisting of water, PBS buffer and TE buffer.

12. The method as claimed in claim 1, wherein the nucleic acid is selected from a group consisting of natural and synthetic nucleic acid.

13. The method as claimed in claim 12, wherein the synthetic nucleic acid is selected from a group consisting of synthetic vector and nucleic acid fragment.

14. A water insoluble medium with nucleic acid, said medium is prepared from the method according to claim 1.

15. The method as claimed in claim 1, further comprising labeling solid article or substance with the third mixture containing said nucleic acid and drying the labeled solid article or substance.

16. The method as claimed in claim 1, further comprising mixing and labeling liquid article or substance with the third mixture containing said nucleic acid.
